(19) United States
(12) Patent Application Publication
(10) Pub. No.: US 2019/0046384 A1
(43) Pub. Date: Feb. 14, 2019

(54) METHOD FOR CONTROLLING A MASSAGE DEVICE AND MASSAGE SYSTEM INCORPORATING SUCH A DEVICE

(71) Applicant: AURASENS, Paris (FR)

(72) Inventor: Olivier ZELLER, Paris (FR)

(21) Appl. No.: 15/758,788

(22) PCT Filed: Sep. 9, 2016
(86) PCT No.: PCT/FR2016/052278

§ 371 Date: Mar. 9, 2018

(30) Foreign Application Priority Data
Sep. 10, 2015 (FR) 1558403

(51) Int. Cl. A61H 1/00; A61H 23/02; A61M 21/02

(52) U.S. Cl. CPC A61H 1/005; A61H 23/0236; A61M 2021/0022; A61H 2023/0209; A61H 2201/0142; A61M 21/02

(57) ABSTRACT

This method makes it possible to control a massage device comprising a plurality of localised tactile elements. It comprises:—a step (E10) of obtaining a first audio stream (FA1);—a step (E50) of obtaining tactile signals (ST) from at least one characteristic extracted from the content of said first audio stream (FA1); each of said tactile signals (ST) being sent (E60) to one of said tactile elements (11) depending on the location of same in order to activate said tactile element for a time period and at an intensity according to said tactile signal, the tactile signals (ST) being obtained in such a way that the tactile elements (11) are activated from one to the next so as to create a massage wave propagating in the massage device.

### BACKGROUND OF THE INVENTION

The invention relates to the field of massage systems or devices. Document US 2011/0245586 published on Oct. 6, 2011 describes a relaxation armchair comprising transducers which can be set vibrating to provide massage to an individual installed in this armchair.

Unfortunately, this armchair does not provide massage similar to what a human masseur could produce.

### OBJECT AND SUMMARY OF THE INVENTION

The aim of the invention is a massage system for performing a variety of massages similar to what a human masseur could produce.

More precisely, the invention relates to a method for controlling a massage device comprising a plurality of localized tactile elements.

In terms of the present application, a tactile element is a controllable element, optionally with variable intensity, and which can be felt by the body. In particular this can be a vibrating element, a heating element, a rolling element, or an element designed to exert pressure.

This method comprises:
a step of obtaining a first audio stream;
a step of extracting at least one characteristic of the contents of this first audio stream;
a step of obtaining tactile signals from said at least one characteristic; each one of the tactile signals being addressed to one of the tactile elements based on its location to activate this tactile element according to a duration and intensity compliant with the tactile signal, the tactile signals being obtained such that the tactile elements are activated in sequence from one tactile element to an adjacent tactile element to create a massage wave propagating in the massage device.

Correlatively, the invention relates to a massage system comprising:
a first massage device comprising a plurality of localized tactile elements;
a module for obtaining a first audio stream; and
a module for obtaining tactile signals configured to extract at least one characteristic of the contents of the first audio stream and for obtaining tactile signals from said at least one characteristic;
a control unit capable of addressing each one of the tactile signals to one of said tactile elements selected based on its location, to activate said tactile element according to a duration and intensity compliant with said tactile signal;
the tactile signals being obtained such that the tactile elements are activated in sequence from one tactile element to an adjacent tactile element to create a massage wave propagating in the first massage device.

In this way and in general, the invention proposes a massage system wherein the tactile elements are controlled to give the user a sensation of continuous movement. These continuous movements can thus reproduce the gestures (movements and pressures) which a masseur could describe using his hands or other parts of his body (fingers, heels, knees, . . . ) or massage objects (rollers, stones, . . . ).

In particular, when the tactile elements are activated in sequence from one tactile element to an adjacent tactile element, just when a tactile element is activated, the adjacent tactile element can be systematically deactivated so as to produce a sensation of displacement.

In a particular embodiment, the tactile elements are activated in sequence from one tactile element to an adjacent tactile element and according to animation determined by the evolution of the extracted characteristic(s). Animation of the tactile elements in particular defines the speed, trajectory and size of the massage wave.

The first massage device can especially consist of a chair, an armchair, a cushion, a mattress, a combination, a jacket, slippers, gloves, a bonnet, a travel pillow or a sexual aid.

In a particular embodiment, the control unit is incorporated into the first massage device. As a variant, all or part of this unit can be placed in remote equipment.

In a particular embodiment of the invention, the method according to the invention comprises a step of restoring auditory signals (or “audio signals”) obtained from the first audio stream in synchronization with the tactile signals. In this embodiment, the system according to the invention comprises a module for generating a sequence of auditory signals from the first audio stream and means for restoring these auditory signals in synchronization with the tactile signals, for example a loudspeaker integrated or not into the first massage device.

The massage system and the method according to the invention thus enable synchronization between the massage waves animated by activation of the different tactile elements and the sound signal (typically music) restored by the loudspeaker of the system.

This synchronization takes the user of the massage system to targeted mental states, for entertainment purposes (sending tactile signals to a body synchronized with video game events), relaxation (massage by vibrations), stimulation or care (synchronization of the sense of touch and hearing for a neurologic awakening).

The system can also serve for receiving information signals on the body in the case of an aircraft pilot, for example.

In a particular embodiment, the method according to the invention further comprises:
a step of creating a video comprising at least one graphic object, this object being animated in the video based on one or more characteristic extracted from the contents of the first audio stream; and
a step of projecting these graphic objects onto a surface on which the tactile elements are shown according to their arrangement in the first massage device, this surface being partitioned into zones, each zone being associated with a tactile element whereof the representation is contained in this zone;
the tactile signal addressed to a tactile element at a given time being obtained from a value representative of the projection of the graphic objects in the zone associated with this tactile element at a time corresponding to said video.

In this embodiment, the system according to the invention comprises:
a module for creating a video comprising at least one graphic object, this object being animated in the video based on at least one characteristic extracted from the contents of the first audio stream; and
a module for projecting these graphic objects onto a surface on which the tactile elements are shown according to their arrangement in the first massage device, this surface being partitioned into zones, each zone being associated with a tactile element whereof the representation is contained in this zone;
the tactile signal addressed to a tactile element at a given time being obtained from a value representative of the projection of the graphic objects in the zone associated with this tactile element at a time corresponding to said video.

Highly advantageously, this embodiment produces a computer tool for creating massages, in which a user can select graphic objects representing massage organs or objects (hands, heels, feet, rollers, stones, . . . ), the massage provided by the system according to the invention then reproducing massage waves similar to those which could have been created by a masseur using organs and objects of the same form.

In a particular embodiment, a change in the nature of the propagation of the massage wave is triggered by a transition in the first audio stream.

This change of nature can especially manifest via a change in speed, trajectory or size of the massage wave.

For example, when a transition is detected, a graphic object can be replaced by another, the animation of a graphic object can be changed, a graphic object or its animation can be added, deleted or modified, where the effect of each one of these operations is to modify the propagation of the massage wave(s) in the device.

In a particular embodiment, the tactile signal addressed to a tactile element at a given time is obtained by multiplying an activation flow by the above value.

This or these activation flows can especially consist of:
a second audio stream
said first audio stream; or
a carrier modulated by the envelope of the first audio stream.

In an embodiment, the massage system further comprises equipment for performing at least one of the following functions:
downloading, obtaining and/or selecting the first audio stream and optionally the activation flow;
downloading, obtaining and/or selecting the tactile signals;
start, pause, stop the restoration of the tactile signals and optionally the restoration of the auditory signals;
adjusting the activation intensity of the tactile elements and optionally the sound volume of the audio signals.

This equipment can for example consist of a remote control or by a smartphone in which an ad-hoc application corresponding to these different functions has been installed.

In an embodiment shown in FIG. 6, the massage system 1′ according to the invention comprises at least one second massage device 10′ directly or indirectly connected to the first massage device 10, said massage system comprising means for addressing at least some of said tactile signals addressed to the tactile elements of the first massage device to tactile elements corresponding of at least one second massage device. As illustrated in FIG. 6, the first massage device can further optionally transmit the audio signals to one or more second massage device(s).

This embodiment chains the massage devices by connecting them together to enable a collective experience. The connection can be made by a wire, by wireless technology (Wifi, bluetooth . . . ) and/or by internet. The connected massage devices could be located in the same space or else be remote from each other.

In a particular embodiment, the first massage device is the master and the second massage devices the slaves. In this mode all the tactile signals sent to the first massage device are sent to the second massage devices.

In another particular embodiment, the control unit of the massage system broadcasts at least some tactile signals sent to the first massage device at zero, one or more second massage devices.

### BRIEF DESCRIPTION OF THE DRAWINGS

Other characteristics and advantages of the present invention will emerge from the following description in reference to the appended drawings which illustrate an exemplary embodiment devoid of any limiting character, in which:

FIG. 1 illustrates a massage system according to the invention in a first particular embodiment;

FIG. 2 schematically illustrates a control unit which can be used in a massage system according to the invention;

FIG. 3 illustrates in flowchart form the main steps of a method for controlling a massage system according to a particular embodiment of the invention;

FIGS. 4A to 4C illustrate examples of animation of graphic objects in an example for creating tactile signals;

FIGS. 5A to 5R illustrate the intensity of the tactile signals sent to the vibrating tactile elements of the massage system of FIG. 1 according to the sequence created in the example of FIGS. 4A to 4C; and

FIG. 6 illustrates a massage system according to the invention in a second particular embodiment.

### DETAILED DESCRIPTION OF AN EMBODIMENT

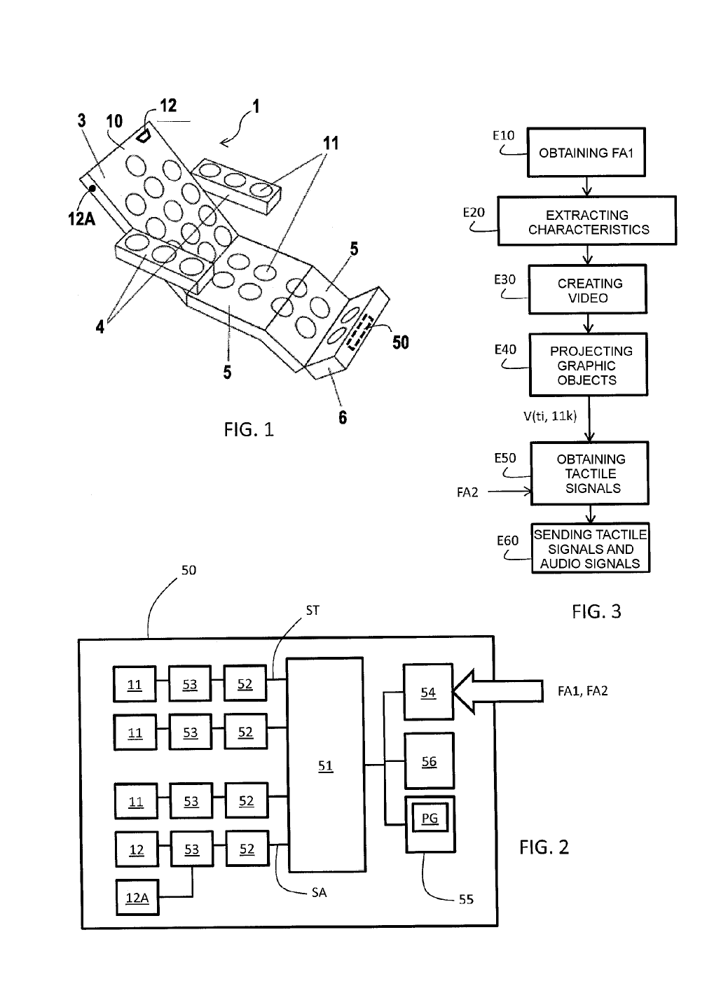

FIG. 1 illustrates a massage system 1 according to a particular embodiment of the invention.

The massage system 1 comprises a massage device 10 comprising a plurality of vibrating tactile elements 11. In the embodiment described here, the massage device consists of a massage armchair, the vibrating tactile elements 11 being localized in the armrests 4, in the footrest 6, in the backrest 3 and in the seat 5 of this armchair.

In the embodiment described here, the massage armchair 10 comprises a loudspeaker 12, located for example in the upper part of the backrest.

In the embodiment described here, the massage armchair 10 also comprises a plug 12A for connecting speakers or headphones.

In the embodiment described here, the massage device comprises a control unit 50, located in the footrest and shown in detail in FIG. 2. This control unit 50 comprises a bus and addressing system for independently sending, to each one of the vibrating tactile elements 11, a tactile signal for making the vibrating tactile element vibrate with a set duration and intensity.

More precisely, the control unit 50 comprises a processor 51 capable of generating tactile signals ST intended for the vibrating tactile elements 11, such a digital signal on output from the processor 51 being converted into an analog signal by a digital/analog converter 52 and amplified by an amplifier 53 on its transmission chain to the relevant vibrating tactile element 11.

In keeping with the invention, the massage system 1 comprises a module for generating the tactile signals ST from one or more characteristics extracted from the contents of a first audio stream FA1.

In the embodiment described here, the massage armchair 10 comprises a module 54 for obtaining this first audio stream FA1, the tactile signals being generated by the processor 51 when it runs the computer program PG which will be described hereinbelow in reference to FIG. 3.

In the embodiment described here, the processor 51 is also capable of generating audio signals SA intended for the loudspeaker 12 and the connecting plug 12A, from the first audio stream FA1.

In the embodiment described here, the control unit 50 comprises a ROM 55 in which the computer program PG is stored and a RAM of type 56 in which variables and registers are stored during execution of the program PG.

In reference to FIG. 3, the main steps of a method for controlling the massage device 10 carried out by the massage system 1 will now be described, this method being in accordance with a particular embodiment of the invention.

During a step E10, the massage system 1 receives a first audio stream FA1. This audio stream can especially consist of a digital audio file, by the audio track of a digital video, by the audio track of a video game, or by an audio stream received streaming via a local network or not. The audio stream FA1 can be received from wired or wireless communication means or be read directly on a memory, for example a USB key.

During a sequence E20, the processor 51 of the massage system 1 extracts one or more characteristics from the contents of the first audio stream FA1. In the embodiment described here, these characteristics consist of:
the tempo of the audio stream;
the transitions between the verses and the chorus;
the frequency of the audio stream;
the detection of a note.

In the embodiment described here, during a step E30, the processor 51 of the massage system creates a video in which graphic objects OG are animated based on the characteristics of the signal extracted in step E20.

In the example described here, the following are used:
a first graphic object OG1 in the form of a bar moving vertically to reproduce the effect of a massage roller, based on the tempo of a chorus;
a second graphic object OG2 in the form of an ellipse whereof the surface varies to reproduce the effect of a more or less warm heating stone, based on the tempo of a verse; and
a third graphic object OG3 in the form of a foot whereof the orientation varies based on the height of the main melody of the audio signal, since a new height of note (in the example re 4, sol 3, re 3) is detected to reproduce the effect of massage with a foot.

In the embodiment described here, the graphic object OG3 can optionally be animated at the same time as the object OG1 or the object OG2.

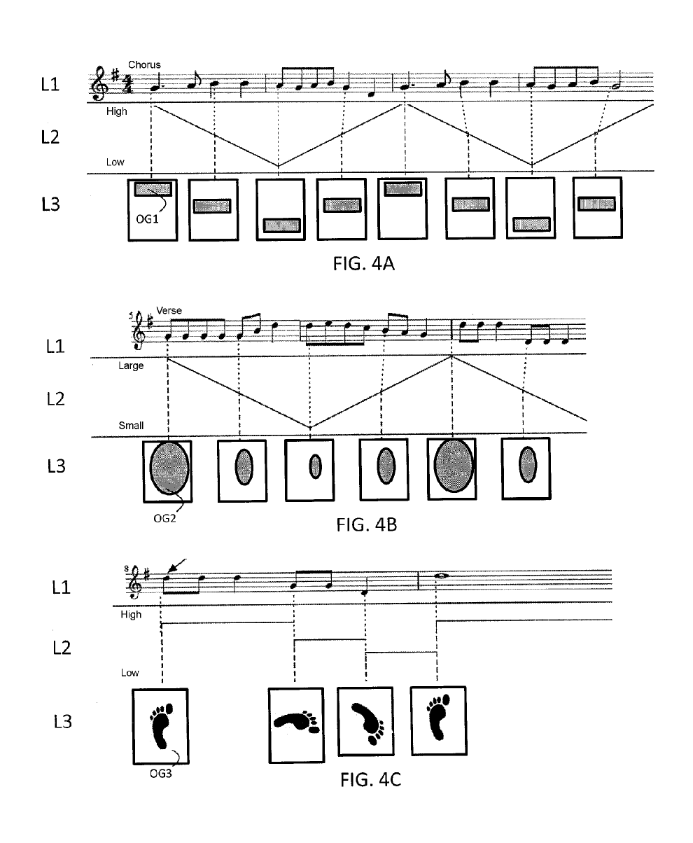

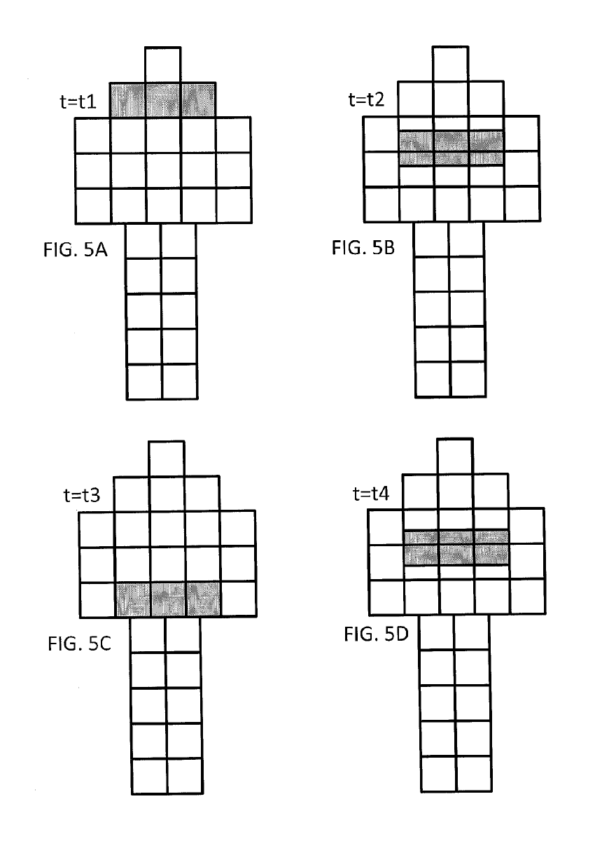

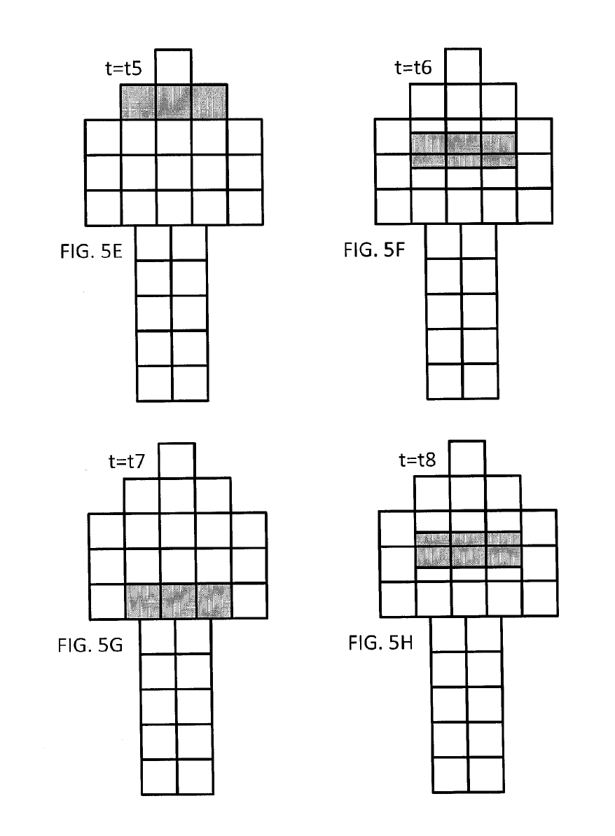

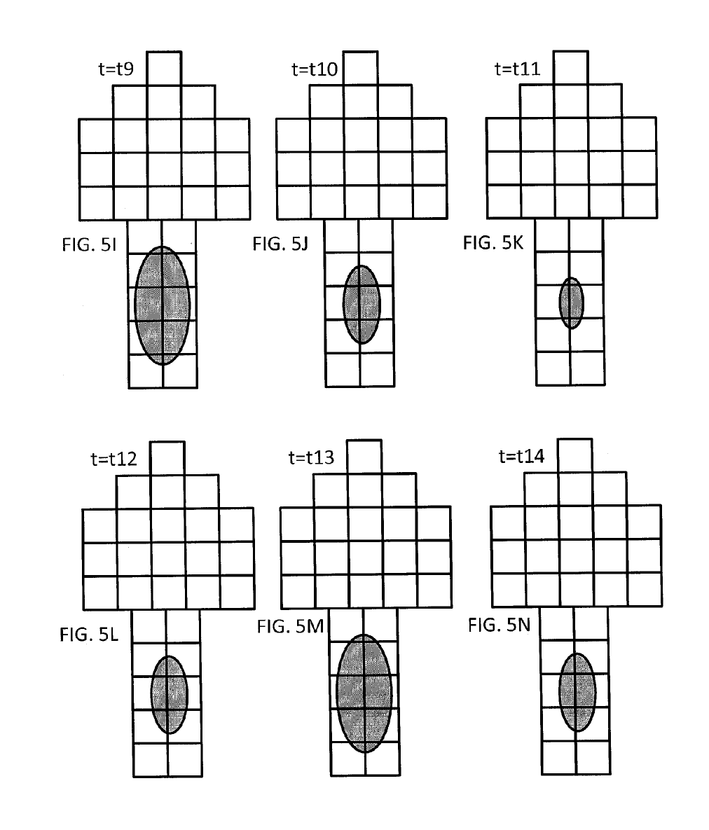

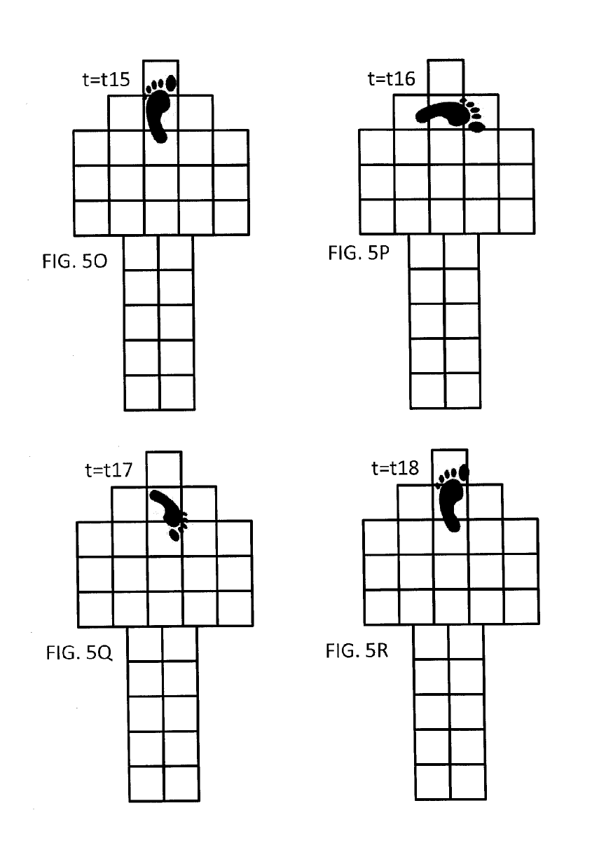

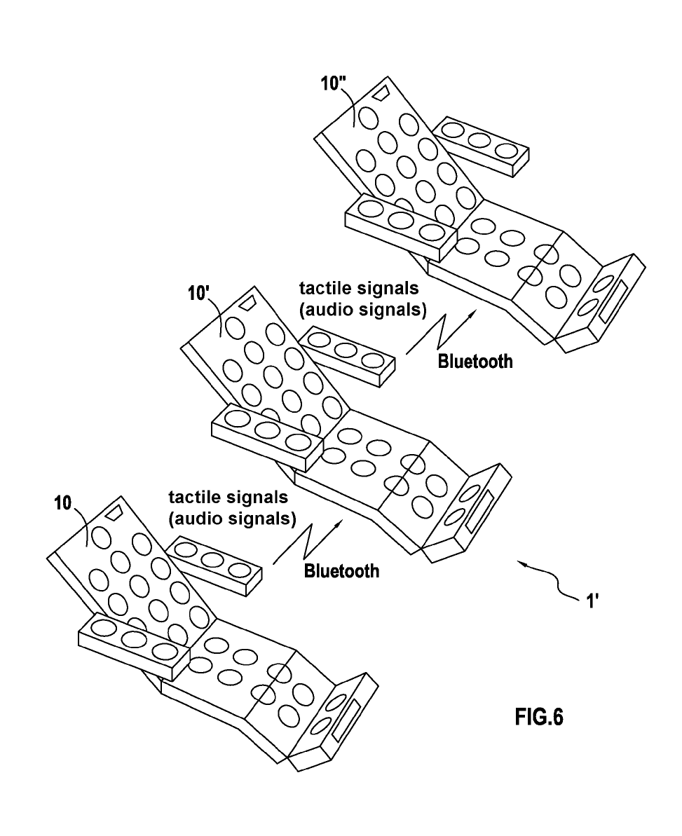

These three examples of animation of graphic objects will now be described in reference to FIGS. 4A to 4C.

In FIG. 4A, line L3 shows the first graphic object OG1 in the form of a rectangle moving vertically based on the tempo of an audio signal corresponding to the partition of a chorus shown in line L1, taken at times t1 to t8 shown in line L2.

In FIG. 4B, line L3 show the second graphic object OG2 in the form of an ellipse whereof the surface varies based on the tempo of an audio signal corresponding to the partition of a verse shown in line L1, taken at times t9 to t14 shown in line L2.

In this example, the graphic object OG1 moves to the graphic object OG2 when the chorus/verse transition is detected.

In FIG. 4C, line L3 shows the third graphic object OG3 in the form of a foot whereof the orientation varies based on the height of the melody of the partition of line L1, taken at times t15 to t18 shown in line L2.

As per this embodiment, the graphic objects OG1 to OG3 are projected during a step E40 onto a surface S on which the vibrating tactile elements 11 are shown according to their arrangement in the device 10, this surface being partitioned into zones, each zone being associated with a vibrating tactile element contained in this zone.

In the embodiment described here, the processor 51 of the massage system S1 generates for each time ti, and each vibrating tactile element 11j, a value V(ti, 11j) corresponding to the percentage of the surface associated with this vibrating tactile element 11 in the surface S covered by a graphic element OG at this time ti.

Therefore by way of example, if it is considered that this value can be between 0 (inactive element) and 100 (vibration with maximal amplification), the values intended for the vibrating tactile element 11 located in the center of the second line, substantially facing the neck of a user lying on the armchair 10 would take the following values:

t

| t1 | t2 | t3 | t4 | t5 | t6 | t7 | t8 | . . . | t14 | t15 | t16 | t17 | t18 |
|---|---|---|---|---|---|---|---|---|---|---|---|---|---|
| 100 | 0 | 0 | 0 | 100 | 0 | 0 | 0 | 0 | 0 | 50 | 50 | 30 | 50 |

In the embodiment described here, these values are multiplied at the step E50 by a second audio stream FA2 to generate the tactile signals ST.

The tactile signals can be recorded in a file in combination and in synchronization with the first audio stream FA1 or be addressed directly, on the fly, to the different vibrating tactile elements.

During a step E60, the tactile signals ST are addressed to the different vibrating tactile elements 11 of the massage device. In the embodiment described here, the first audio stream FA1 is sent to the loudspeaker 12.

Amplification of the different vibrating tactile elements 11 of the massage device 10 thus follows continuous displacement of the graphic objects OG1 to OG3. They restore one or more massage waves which propagate in the massage device based on the evolution of characteristics of the contents of the first audio stream FA1, in synchronization with the audio stream FA1 restored by the loudspeaker.

More particularly, in the example described previously:
a first massage wave follows the tempo of the chorus of the audio stream FA1;
a second massage wave follows the tempo of the verse of the audio stream FA1; and
a third massage wave follows the frequency of the main melody of the first audio stream FA1.

In the example described hereinabove, the graphic object OG1 moves to the graphic object OG2 during detection of a chorus/verse transition.

As a variant, other transitions can be used, especially:
the appearance of a note, a chord or a silence;
the appearance or disappearance of an instrument or a voice;
a change of section, for example an introduction, verse, pre-chorus, chorus, middle part, solo, conclusion, improvisation;
a silence;
a change of piece;
a transposition;
a change of tonality;
a change of tempo;
a change of rhythmic signature;
a change of intensity for a frequency band, representative for example of the application of a filter;
the application of an effect (reverb, chorus, slicer, . . . );
a change of intensity;
a change of balance.

In the embodiment previously describe, the same second audio stream FA2 has been used to generate all the tactile signals.

As a variant, different audio stream FA2, FA3, . . . (second audio stream in terms of the invention) can be used for different graphic objects.

1. A method for controlling a massage device comprising a plurality of tactile elements, the method comprising:
obtaining a first audio stream;
extracting at least one characteristic of the contents of the first audio stream; and
obtaining tactile signals from said at least one characteristic;
each one of said tactile signals being addressed to one of said plurality of tactile elements based on its location to activate said one of said plurality of tactile elements according to a duration and an intensity compliant with said one of said tactile signals, and
the tactile signals being obtained such that the plurality of tactile elements are activated in sequence from one tactile element to an adjacent tactile element to create a massage wave propagating in the massage device.

2. The method according to claim 1, wherein the plurality of tactile elements are activated in sequence from one tactile element to an adjacent tactile element and according to animation determined by an evolution of said at least one extracted characteristic.

3. The method according to claim 1, wherein a change in a nature of the propagating of the massage wave is triggered by a transition in the first audio stream.

4. The method according to claim 1, further comprising: restoring auditory signals obtained from said first audio stream in synchronization with said tactile signals.

5. The method according to claim 1, further comprising:
creating a video comprising at least one graphic object, said at least one graphic object being animated in the video based on said at least one characteristic; and
projecting said at least one graphic object onto a surface on which said plurality of tactile elements are shown according to their arrangement in the massage device, said surface being partitioned into zones, each zone being associated with a tactile element whereof the representation is contained in this zone;
the tactile signal addressed to a tactile element at a given time being obtained from a value representative of the projecting of said at least one graphic object in the zone associated with the tactile element at a time corresponding to said video.

6. The method according to claim 5, wherein said tactile signal addressed to a tactile element at a given time is obtained by multiplying an activation flow by said value.

7. The method according to claim 6, wherein the activation flow comprises:
a second audio stream;
said first audio stream; or
a carrier modulated by an envelope of the first audio stream.

8. A massage system comprising:
a first massage device comprising a plurality of tactile elements;
a module for obtaining a first audio stream; and
a module for obtaining tactile signals, which is configured to extract at least one characteristic of the contents of said first audio stream and for obtaining tactile signals from said at least one characteristic;
a control unit capable of addressing each one of said tactile signals to one of said plurality of tactile elements selected based on its location to activate said one of said plurality of tactile elements according to a duration and an intensity compliant with said one of said tactile signals;
the tactile signals being obtained such that the plurality of tactile elements are activated in sequence from one tactile element to an adjacent tactile element to create a massage wave propagating in said first massage device.

9. The system according to claim 8, further comprising:
a module for generating a sequence of auditory signals from said first audio stream; and
means for restoring said sequence of auditory signals in synchronization with said tactile signals.

10. The system according to claim 8 further comprising:
a module for creating a video comprising at least one graphic object, said at least one graphic object being animated in the video based on said at least one characteristic; and
a module for projecting said at least one graphic object onto a surface on which said plurality of tactile elements are shown according to their arrangement in the first massage device, said surface being partitioned into zones, each zone being associated with a tactile element whereof the representation is contained in this zone;
the tactile signal addressed to a tactile element at a given time being obtained from a value representative of the projecting of said at least graphic object in the zone associated with the tactile element at a time corresponding to said video.

11. The system according to claim 10, wherein said tactile signal addressed to a tactile element at a given time is obtained by multiplying an activation flow by said value.

12. The massage system according to claim 8, wherein said first massage device comprises a chair, an armchair, a cushion, a mattress, a combination, a jacket, slippers, gloves, a bonnet or a sexual aid.

13. The massage system according to claim 8, further comprising equipment for performing at least one of the following functions:
obtaining the first audio stream and optionally an activation flow;
obtaining the tactile signals;
controlling restoration of the tactile signals and optionally restoration of the auditory signals; and
adjusting the activation intensity of the plurality of tactile elements and optionally a sound volume of the first audio stream.

14. The massage system according to claim 8, further comprising:
at least one second massage device directly or indirectly connected to said first massage device, and
means for addressing at least some of said tactile signals addressed to the plurality of tactile elements of the first massage device to tactile elements corresponding to the at least one second massage device.
